(19) United States
(12) Patent Application Publication
(10) Pub. No.: US 2024/0366298 A1
(43) Pub. Date: Nov. 7, 2024

(54) Flexible Telescopic Shaft for Monopolar Electrosurgical Pencil

(71) Applicant: Stryker European Operations Limited, Cork (IE)

(72) Inventors: Micheal Burke, Cork (IE); Scott McFarland, Greenisland (IE); Paul Sheridan, Wexford (IE); Laura Constance Frey, Belfast (IE)

(21) Appl. No.: 18/686,272

(22) PCT Filed: Aug. 26, 2022
(86) PCT No.: PCT/IB2022/000484

§ 371 Date: Feb. 23, 2024

Related U.S. Application Data
(60) Provisional application No. 63/237,753, filed on Aug. 27, 2021.

(51) Int. Cl. A61B 18/14; A61B 17/00; A61B 18/00

(52) U.S. Cl. CPC A61B 18/1492; A61B 2017/00526; A61B 2018/00077; A61B 2018/00178; A61B 2218/008

(57) ABSTRACT

An electrosurgical device includes a handle and a shaft extending from the handle to define a distal end of the electrosurgical device. The electrosurgical device includes one or more electrical elements coupled to the shaft at the distal end of the electrosurgical device. The shaft is configured to bend and position the one or more electrical elements relative to the handle. The shaft includes an outer structure defining a passageway extending from the handle to the one or more electrical elements. The shaft includes one or more conductors extending through the passageway to transmit electrical signals from an electrosurgical generator to the one or more electrical elements. The one or more conductors form a coil configured to bend in response to a force that exceeds a threshold and to determine a bended shape for the shaft.

### CROSS-REFERENCE TO RELATED APPLICATION

This application claims the benefit of U.S. Provisional Application No. 63/237,753, filed Aug. 27, 2021, the contents of which are hereby incorporated by reference in their entirety.

### BACKGROUND

Electrosurgery generally refers to the use of electricity to cause thermal destruction of biological tissue. For instance, in an approach known as high-frequency electrosurgery, high-frequency alternating current is applied to tissue and converted to heat by a resistance associated with the tissue. Electrosurgery may be employed in medical procedures, such as cardiothoracic, neurological, gynecological, orthopedic, cosmetic, or dental procedures, where tissue is fulgurated, coagulated, cut, and/or dissected.

In bipolar electrosurgery, an electrosurgical device includes an active electrode and a return electrode. For instance, the two tips of a forceps respectively perform the active and return electrode functions at a surgical site. The tissue grasped between the two tips is a part of the electrical circuit defined by the forceps and is heated by the current passing between the two tips.

In monopolar electrosurgery, an electrosurgical device includes an active electrode that is placed at a surgical site. A return electrode is placed somewhere else on the body of the patient. The current passes through the patient from the active electrode to the return electrode and generates heat at the surgical site. For instance, an electrosurgical pencil is a device that includes an electrode tip that can be applied to a surgical site for monopolar electrosurgery. The electrode tip may be configured to have a blade, needle, ball, or other shape depending on the particular procedure.

### SUMMARY

An electrosurgical device, such as an electrosurgical pencil, includes an electrode disposed on a flexible, telescopic shaft. The shaft is sufficiently flexible to take different shapes that allow the electrode to be maneuvered around obstructions and/or through small spaces and to reach desired surgical sites. Additionally, the shaft is sufficiently rigid to maintain a desired shaped during maneuvering and allow precise positioning of the electrode for electrosurgery: The shaft can extend deep into a body and allow the electrosurgical device to apply electrosurgery at surgical sites that would be otherwise too difficult to reach. The ability to reach such surgical sites with the shaft allows the size of surgical incisions and resulting scars to be reduced.

According to an example implementation, an electrosurgical device includes a handle and a shaft extending from the handle to define a distal end of the electrosurgical device. The electrosurgical device includes one or more electrical elements coupled to the shaft at the distal end of the electrosurgical device. The shaft is configured to bend and position the one or more electrical elements relative to the handle. The shaft includes an outer structure defining a passageway extending from the handle to the one or more electrical elements. The shaft includes one or more conductors extending through the passageway to transmit electrical signals from an electrosurgical generator to the one or more electrical elements. The one or more conductors form a coil configured to bend in response to a force that exceeds a threshold and to determine a bended shape for the shaft.

According to another example implementation, an electrosurgical device includes a handle and a shaft extending from the handle to define a distal end of the electrosurgical device. The electrosurgical device includes one or more electrical elements coupled to the shaft at the distal end of the electrosurgical device. The shaft is configured to bend and position the one or more electrical elements relative to the handle. The shaft includes an outer structure defining a passageway extending from the handle to the one or more electrical elements. The shaft includes one or more conducting wires extending through the passageway to provide electrical signals from an electrosurgical generator to the one or more electrical elements. The one or more conducting wires form a shape configured to bend in response to a force that exceeds a threshold and to determine a bended shape for the shaft.

According to a further example implementation, an electrosurgical device includes a handle and a shaft extending from the handle to define a distal end of the electrosurgical device. The electrosurgical device includes one or more electrical elements coupled to the shaft at the distal end of the electrosurgical device. The shaft is configured to bend in response to a force that exceeds a threshold and thereby reposition the one or more electrical elements relative to the handle. The shaft includes an outer structure defining a passageway extending from the handle to the one or more electrical elements. The shaft includes an inner support structure disposed within the outer structure and configured to bend with the outer structure and transversely support the outer structure from within and along the passageway. One or more conductors are integrated into the inner support structure. The one or more conductors are configured to provide electrical signals from an electrosurgical generator to the one or more electrical elements.

The features, functions, and advantages that have been discussed can be achieved independently in various embodiments or may be combined in yet other embodiments further details of which can be seen with reference to the following description and figures.

### BRIEF DESCRIPTION OF THE FIGURES

The novel features believed characteristic of the illustrative embodiments are set forth in the appended claims. The illustrative embodiments, however, as well as a preferred mode of use, further objectives and descriptions thereof, will best be understood by reference to the following detailed description of an illustrative implementation of the present disclosure when read in conjunction with the accompanying figures, wherein:

FIG. 1 depicts a simplified block diagram of an electrosurgical system, according to an example.

FIG. 2 depicts an electrosurgical device, according to an example.

FIG. 3A depicts another electrosurgical device, according to an example.

FIG. 3B depicts an assembly view of the shaft for the electrosurgical device of FIG. 3A.

FIG. 3C depicts aspects of the shaft for the electrosurgical device of FIG. 3A.

FIG. 3D depicts further aspects of the shaft for the electrosurgical device of FIG. 3A.

FIG. 4 depicts a flowchart of a method of operating an electrosurgical device, according to an example.

FIG. 5 depicts a flowchart of a method of operating an electrosurgical device that can be used with the method of FIG. 4, according to an example.

FIG. 6 depicts a flowchart of a method of operating an electrosurgical device that can be used with the method of FIG. 4, according to an example.

FIG. 7 depicts a flowchart of a method of forming an electrosurgical device, according to an example.

FIG. 8 depicts a flowchart of a method of forming an electrosurgical device that can be used with the method of FIG. 7, according to an example.

### DESCRIPTION

Disclosed examples will now be described more fully hereinafter with reference to the accompanying drawings, in which some, but not all of the disclosed examples are shown. Indeed, several different examples may be described and should not be construed as limited to the examples set forth herein. Rather, these examples are described so that this disclosure will be thorough and complete and will fully convey the scope of the disclosure to those skilled in the art.

By the term “approximately” or “substantially” with reference to amounts or measurement values described herein, it is meant that the recited characteristic, parameter, or value need not be achieved exactly, but that deviations or variations, including for example, tolerances, measurement error, measurement accuracy limitations and other factors known to those of skill in the art, may occur in amounts that do not preclude the effect the characteristic was intended to provide.

As disclosed herein, electrosurgical devices, such as electrosurgical pencils, employ shafts that are sufficiently flexible to take different shapes, but sufficiently rigid to maintain a selected shape. As such, an electrosurgical electrode disposed at a distal end of the shaft can be maneuvered around obstructions and/or through small spaces and precisely positioned at desired surgical sites. The shaft, which may also be telescoping, can extend deep into a body and allow the electrosurgical device to apply electrosurgery at surgical sites that would be otherwise too difficult to reach. The ability to reach such surgical sites with the shaft allows the size of surgical incisions and resulting scars to be reduced. In some examples, conductors for one or more electrical elements extend through the shaft and define an inner structure to help provide these advantageous structural characteristics.

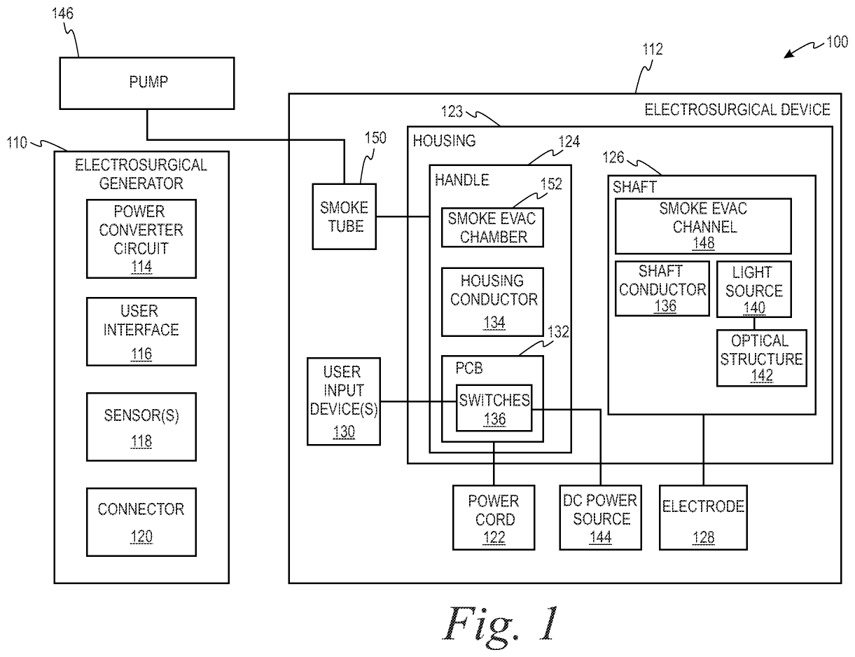

Referring to FIG. 1, an electrosurgical system 100 is shown according to an example. As shown in FIG. 1, the electrosurgical system 100 includes an electrosurgical generator 110 and an electrosurgical device 112. In general, the electrosurgical generator 110 can generate electrosurgical energy that is suitable for performing electrosurgery on a patient. For instance, the electrosurgical generator 110 can include a power converter circuit 114 that can convert a grid power to electrosurgical energy such as, for example, a radio frequency (RF) output power. As an example, the power converter circuit 114 can include one or more electrical components (e.g., one or more transformers) that can control a voltage, a current, and/or a frequency of the electrosurgical energy.

Within examples, the electrosurgical generator 110 can include a user interface 116 that can receive one or more inputs from a user and/or provide one or more outputs to the user. As examples, the user interface 116 can include one or more buttons, one or more switches, one or more dials, one or more keypads, one or more touchscreens, and/or one or more display screens.

In an example, the user interface 116 can be operable to select a mode of operation from among a plurality of modes of operation for the electrosurgical generator 110. As examples, the modes of operation can include a cutting mode, a coagulating mode, an ablating mode, and/or a sealing mode. Combinations of these waveforms can also be formed to create blended modes. In one implementation, the modes of operation can correspond to respective waveforms for the electrosurgical energy. As such, in this implementation, the electrosurgical generator 110 can generate the electrosurgical energy with a waveform selected from a plurality of waveforms based, at least in part, on the mode of operation selected using the user interface 116.

The electrosurgical generator 110 can also include one or more sensors 118 that can sense one or more conditions related to the electrosurgical energy and/or the target tissue. As examples, the sensor(s) 118 can include one or more current sensors, one or more voltage sensors, one or more temperature sensors, and/or one or more bioimpedance sensors. Within examples, the electrosurgical generator 110 can additionally or alternatively generate the electrosurgical energy with an amount of electrosurgical energy (e.g., an electrical power) and/or a waveform selected from among the plurality of waveforms based on one or more parameters related to the condition(s) sensed by the sensor(s) 118.

In one example, the electrosurgical energy can have a frequency that is greater than approximately 100 kilohertz (kHz) to reduce (or avoid) stimulating a muscle and/or a nerve near the target tissue. In another example, the electrosurgical energy can have a frequency that is between approximately 300 KHz and approximately 500 KHz.

In FIG. 1, the electrosurgical generator 110 also includes a connector 120 that can facilitate coupling the electrosurgical generator 110 to the electrosurgical device 112. For example, the electrosurgical device 112 can include a power cord 122 having a plug, which can be coupled to a socket of the connector 120 of the electrosurgical generator 110. In this arrangement, the electrosurgical generator 110 can supply the electrosurgical energy to the electrosurgical device 112 via the coupling between the connector 120 of the electrosurgical generator 110 and the power cord 122 of the electrosurgical device 112.

As shown in FIG. 1, the electrosurgical device 112 can include a housing 123. The housing 123 can be an elongated structure in and/or on which components of the electrosurgical device 112 can be disposed. In some examples, the housing 123 can be an integral, monolithic structure. In other examples the housing 123 can include a plurality of structures that are coupled to each other.

In FIG. 1, the housing 123 includes a handle 124 that defines an interior bore, a shaft 126 extending in a distal direction from the handle 124, and an electrosurgical electrode 128 coupled to the shaft 126. In general, the handle 124 can be configured to facilitate a user gripping and manipulating the electrosurgical device 112 while performing electrosurgery. For example, the handle 124 can have a shape and/or a size that can facilitate a user performing electrosurgery by manipulating the electrosurgical device 112 using a single hand. In one implementation, the handle 124 can have a shape and/or a size that facilitates the user holding the electrosurgical device 112 in a writing utensil gripping manner (e.g., the electrosurgical device 112 can be an electrosurgical pencil).

Additionally, for example, the handle 124 and/or the shaft 126 can be constructed from one or more materials that are electrical insulators (e.g., a plastic material). This can facilitate insulating the user from the electrosurgical energy flowing through the electrosurgical device 112 while performing the electrosurgery.

In some implementations, the shaft 126 can be coupled to the handle 124 in a fixed and non-moveable manner. This may simplify manufacturing and reduce a cost of manufacture. In other implementations, the shaft 126 can be telescopically moveable relative to the handle 124. For example, the shaft 126 can be telescopically moveable in the interior bore defined by the handle 124 to extend the shaft 126 in the distal direction and retract the shaft 126 in a proximal direction relative to the handle 124 (e.g., movable along a longitudinal axis of the electrosurgical device 112). As noted above, the electrosurgical electrode 128 is coupled to the shaft 126 and, thus, the electrosurgical electrode 128 moves together with the shaft 126 in an axial direction along the longitudinal axis relative to the handle 124. This can provide for adjusting a length of the electrosurgical device 112, which can facilitate performing electrosurgery at a plurality of different depths within tissue (e.g., due to different anatomical shapes and/or sizes of patients) and/or at a plurality of different angles.

The electrosurgical electrode 128 can additionally or alternatively be rotatable about an axis of rotation that is parallel to the longitudinal axis of the electrosurgical device 112. In some examples, the electrosurgical electrode 128 can be rotatable relative to the handle 124 and the shaft 126. In other examples, the electrosurgical electrode 128 can be rotationally fixed relative to the shaft 126 such that the shaft 126 and the electrosurgical electrode 128 are rotatable together relative to the handle 124 and at least one additional component in an inner cavity defined by the shaft 126. Rotating the electrosurgical electrode 128 relative to the handle 124 can facilitate adjusting an angle of the electrosurgical electrode 128 relative to one or more user input device(s) 130 of the electrosurgical device 112. In this arrangement, a user can comfortably grip the handle 124 in a position in which their fingers can comfortably operate the user input device(s) 130 while the electrosurgical electrode 128 is set at a rotational position selected from among a plurality of rotational positions relative to the handle 124 based on, for example, a location, a size, and/or a shape of a surgical site in which the user is operating.

In one implementation, the electrosurgical electrode 128 can be rotatable by more than 360 degrees relative to the handle 124. This can improve an ease of use by allowing an operator to freely rotate the electrosurgical electrode 128 without limitation. However, in other implementations, the electrosurgical electrode 128 can be rotatable by less than or equal to 360 degrees (e.g., rotatable by 180 degrees or rotatable by 360 degrees). This may still allow an operator to achieve a desired rotational arrangement, but with the possibility that the operator may rotate in first direction, reach a stop limiting further rotation, and then rotate back in a second direction to achieve the desired rotational arrangement.

The user input device(s) 130 can select between the modes of operation of the electrosurgical device 112 and/or the electrosurgical generator 110. For instance, in one implementation, the user input device(s) 130 can be configured to select between a cutting mode of operation and a coagulation mode of operation. Responsive to actuation of the user input device(s) 130 of the electrosurgical device 112, the electrosurgical device 112 can (i) receive the electrosurgical energy with a level of power and/or a waveform corresponding to the mode of operation selected via the user input device(s) 130 and (ii) supply the electrosurgical energy to the electrosurgical electrode 128.

In FIG. 1, the electrosurgical device 112 includes a plurality of electrical components that facilitate supplying the electrosurgical energy, which the electrosurgical device 112 receives from the electrosurgical generator 110, to the electrosurgical electrode 128. For example, the electrosurgical device 112 can include at least one electrical component selected from a group of electrical components including: a printed circuit board 132 (e.g., a flexible printed circuit board), a housing conductor 134, and/or a shaft conductors 136 that can provide a circuit for conducting the electrosurgical energy from the power cord 122 to the electrosurgical electrode 128. One or more of the electrical components can be positioned in the inner bore defined by the handle 124 and/or in the inner cavity defined by the shaft 126.

Within examples, the user input device(s) 130 can include one or more buttons on an exterior surface of the handle 124. Each button of the user input device(s) 130 can be operable to actuate a respective one of a plurality of switches 138 of the printed circuit board 132. In general, the switches 138 and/or the printed circuit board 132 are operable to control a supply of the electrosurgical energy from the electrosurgical generator 110 to the electrosurgical electrode 128. For instance, in one implementation, when each button is operated (e.g., depressed), the respective switch 138 associated with the button can be actuated to cause the printed circuit board 132 to transmit a signal to the electrosurgical generator 110 and cause the electrosurgical generator 110 to responsively supply the electrosurgical energy with a level of power and/or a waveform corresponding to a mode of operation associated with the button. In another implementation, operating the button and thereby actuating the respective switch 138 associated with the button can close the switch 138 to complete a circuit to the electrosurgical generator 110 to cause the electrosurgical generator 110 to responsively supply the electrosurgical energy with a level of power and/or a waveform corresponding to a mode of operation associated with the button. In some examples of this implementation, the printed circuit board 132 can be omitted.

In both example implementations, the electrosurgical energy supplied by the electrosurgical generator 110 can be supplied from (i) the power cord 122, the printed circuit board 132, and/or the switches 138 to (ii) the electrosurgical electrode 128 by the housing conductor 134 and the shaft conductor 136. As such, as shown in FIG. 1, the printed circuit board 132 can be coupled to the power cord 122, the housing conductor 134 can be coupled to the printed circuit board 132 and the shaft conductor 136, and the shaft conductor 136 can be coupled to the electrosurgical electrode 128. In this arrangement, the housing conductor 134 can conduct the electrosurgical energy (supplied to the housing conductor 134 via the printed circuit board 132) to the shaft conductor 136, and the shaft conductor 136 can conduct the electrosurgical energy to the electrosurgical electrode 128.

In general, the housing conductor 134 and the shaft conductor 136 can each include one or more electrically conductive elements that provide an electrically conductive bus for supplying the electrosurgical energy to the electrosurgical electrode 128. More particularly, the housing conductor 134 can include one or more electrically conductive elements of the handle 124 that can supply the electrosurgical energy to the shaft conductor 136, and the shaft conductor 136 can include one or more electrically conductive elements of the shaft 126 that can supply the electrical energy from the housing conductor 134 to the electrosurgical electrode 128. As described in further detail below; the housing conductor 134 can engage the shaft conductor 136 to maintain an electrical coupling between the housing conductor 134, the shaft conductor 136, and the electrosurgical electrode 128 while (i) the shaft 126 and/or the electrosurgical electrode 128 telescopically moves relative to the handle 124, and/or (ii) the electrosurgical electrode 128 rotates relative to the handle 124.

As shown in FIG. 1, the electrosurgical device 112 can additionally include one or more light sources 140 that are configured to emit light. The light source(s) 140 can be optically coupled to an optical lens assembly 142, which is configured to receive the light emitted by the light source(s) 140 and transmit the light in a distal direction toward a surgical site to illuminate the surgical site while performing electrosurgery using the electrosurgical electrode 128. As described in further detail below; the optical lens assembly 142 can help to direct the light emitted by the light source 140 in the distal direction and thereby improve a quality of the light illuminating the surgical site.

In FIG. 1, the light source 140 can be coupled to the shaft 126. As such, the light source 140 can also move telescopically with the shaft 126 relative to the handle 124. However, in other examples, the light source 140 can be in the interior bore of the handle 124 and/or coupled to an exterior surface of the handle 124. As examples, the light source 140 can include one or more light emitting diodes (LEDs), organic light emitting diodes (OLEDs), optical fibers, non-fiber optic waveguides, and/or lenses. Additionally, for example, the light source 140 can include a light-emitting diode printed circuit board (LED PCB) having one or more light sources (e.g., LEDs). As described in further detail below; the LED PCB can include a PCB aperture, and one or more other components (e.g., the electrosurgical electrode 128) of the electrosurgical device 112 can extend through the aperture.

In implementations that include the light source 140, the user input device(s) 130, the printed circuit board 132, the switches 138, the housing conductor 134, and/or the shaft conductor 136 can additionally supply an electrical power from a direct current (DC) power source 144 to the light source 140. In one example, the DC power source 144 can include a battery disposed in the handle 124, the plug of the power cord 122, and/or a battery receptacle located along the power cord 122 between the handle 124 and the plug. Although the electrosurgical device 112 includes the DC power source 144 in FIG. 1, the DC power source 144 can be separate and distinct from the electrosurgical device 112 in other examples. For instance, in another example, the electrosurgical generator 110 can include the DC power source 144.

Additionally, in implementations that include the light source 140, the user input device(s) 130 can be operable to cause the light source 140 to emit the light. In one example, the user input device(s) 130 can include a button that independently controls the light source 140 separate from the button(s) that control the electrosurgical operational modes of the electrosurgical device 112. In another example, the user input device(s) 130 and the printed circuit board 132 can be configured such that operation of the button(s) that control the electrosurgical operational mode simultaneously control operation of the light source 140 (e.g., the light source 140 can be automatically actuated to emit light when a button is operated to apply the electrosurgical energy at the electrosurgical electrode 128).

As shown in FIG. 1, responsive to operation of the user input device(s) 130 to actuate the light source 140, the DC power source 144 can supply the electrical power (e.g., a DC voltage) to the light source 140 via the printed circuit board 132, the housing conductor 134, and/or the shaft conductor 136. In this implementation, one or more of the conductive elements of the housing conductor 134 can be configured to supply the electrical power from the DC power source 144 to the light source 140 and/or return the electrical power from the light source 140 to the DC power source 144. Accordingly, the housing conductor 134 can additionally or alternatively assist in providing electrical communication between the DC power source 144 and the light source 140 as the shaft 126 and the light source 140 telescopically move relative to the handle 124.

Although the user input device(s) 130 on the handle 124 can be operated to control the operation of the light source 140 in the examples described above, the light source 140 can be additionally or alternatively operated by one or more user input device(s) on the electrosurgical generator 110 (e.g., via the user interface 116) and/or on the plug of the power cord 122.

As noted above, the electrosurgical device 112 can additionally include features that provide for evacuating surgical smoke from a target tissue to a location external to the surgical site. Surgical smoke is a by-product of various surgical procedures. For example, during surgical procedures, surgical smoke may be generated as a by-product of electrosurgical units (ESU), lasers, electrocautery devices, ultrasonic devices, and/or other powered surgical instruments (e.g., bones saws and/or drills). In some instances, the surgical smoke may contain toxic gases and/or biological products that result from a destruction of tissue. Additionally, the surgical smoke may contain an unpleasant odor. For these and other reasons, many guidelines indicate that exposure of surgical personnel to surgical smoke should be reduced or minimized.

To reduce (or minimize) exposure to surgical smoke, a smoke evacuation system may be used during the surgical procedure. In general, the smoke evacuation system may include a suction pump 146 that can generate sufficient suction and/or vacuum pressure to draw the surgical smoke away from the surgical site. In some implementations, the smoke evacuation system may be coupled to an exhaust system (e.g., an in-wall exhaust system) that exhausts the surgical smoke out of an operating room. In other implementations, the smoke evacuation system may filter air containing the surgical smoke and return the air to the operating room. Within examples, the suction pump 146 and the electrosurgical generator 110 can be provided as separate devices or integrated in a single device (e.g., in a common housing).

As shown in FIG. 1, the shaft 126 can include a smoke evacuation channel 148 in the inner cavity of the shaft 126. The smoke evacuation channel 148 can also include a smoke inlet that can extend circumferentially around a center axis of a distal portion of the electrosurgical electrode 128. In this arrangement, the smoke inlet of the smoke evacuation channel can help to receive surgical smoke into the smoke evacuation channel 148 in all rotational alignments of the electrosurgical electrode 128 relative to the handle 124 and/or the electrosurgical device 112 relative to the target tissue. However, in another example, the smoke evacuation channel 148 can include one or more smoke inlets that do not extend circumferentially around the electrosurgical electrode 128.

In an example, the smoke evacuation channel 148 can include an outer tube that is separated from the optical lens assembly 142 by an air gap. For instance, the shaft 126 can include a plurality of standoffs that extend between the optical lens assembly 142 and the outer tube of the smoke evacuation channel 148 to provide the air gap between the outer tube and the optical lens assembly 142. In one implementation, the optical lens assembly 142 can include the standoffs such that the optical lens assembly 142 and the standoffs are formed as a single, monolithic structure. In another implementation, the standoffs can be formed as a single, monolithic structure with the outer tube of the smoke evacuation channel 148. In another implementation, the standoffs can be separate from the outer tube of the smoke evacuation channel 148 and the optical lens assembly 142.

In an example, the electrosurgical device 112 is an electrosurgical pencil, where the electrosurgical electrode 128 is an electrode tip disposed on the shaft 126. In this example, the shaft 126 is sufficiently flexible to take different shapes that allow the electrosurgical electrode 128 to be maneuvered around obstructions and/or through small spaces and to reach desired surgical sites. Additionally, the shaft 126 is sufficiently rigid to maintain a desired shape during maneuvering and allow precise positioning of the electrosurgical electrode 128 for electrosurgery: The shaft 126 can extend deep into a body and allow the electrosurgical device 112 to apply electrosurgery at surgical sites that would be otherwise too difficult to reach. The ability to reach such surgical sites with the shaft 126 allows the size of surgical incisions and resulting scars to be reduced. The shaft 126 may be telescoping to help it extend to such surgical sites, but in some implementations, the shaft 126 can be non-telescoping but have a length that helps it to extend to such surgical sites.

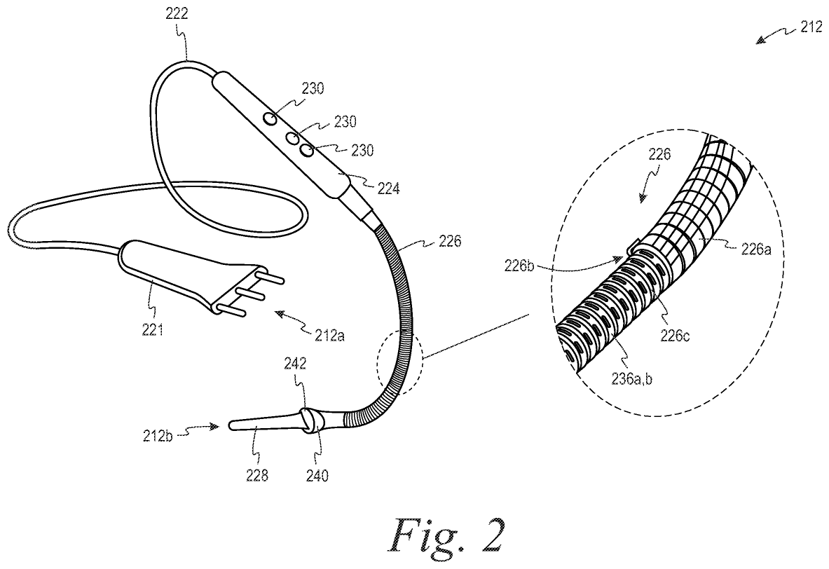

Referring to FIG. 2, an electrosurgical device 212 includes a handle 224 that allows a practitioner to manipulate the electrosurgical device 212. The electrosurgical device 212 includes a shaft 226 extending from the handle 224 to define a distal end 212b of the electrosurgical device 212. The electrosurgical device 212 includes one or more electrical elements coupled to the shaft 226 at the distal end 212b.

As shown in detail in FIG. 2, the shaft 226 includes an outer structure 226a defining a passageway 226b extending from the handle 224 to the one or more electrical elements. The shaft 226 also includes an inner support structure 226c disposed within the outer structure 226a. The inner support structure 226c is configured to bend with the outer structure 226a and transversely support the outer structure 226a from within and along the passageway 226b.

With the combination of the outer structure 226a and the inner support structure 226c, the shaft 226 is configured to bend in response to a force that exceeds a threshold and thereby reposition the one or more electrical elements relative to the handle 224. The shaft 226 is sufficiently flexible to take different shapes (e.g., a bended shape chosen by the practitioner), but is also sufficiently rigid to maintain a desired shaped during maneuvering and operation of the electrosurgical device 212. In some implementations, in comparison to the outer structure 226a, the inner support structure 226c provides greater resistance to bending.

As illustrated, the outer structure 226a may be a coil defined by a series of loops configured to allow bending of the shaft 226, and the inner support structure 226c may be a coil defined by a series of loops configured to bend in response to the force that exceeds the threshold and to determine a bended shape for the shaft 226. Alternatively, the outer structure 226a may be formed from a heat shrink material (e.g., a plastic) configured to shrink over the inner support structure 226c (as a sheath).

In some implementations, the shaft 226 is further configured to move relative to the handle 224 from a retracted position to an extended position. As such, the shaft 226 extends the distal end 212b of the electrosurgical device 212 to a greater distance from the handle 224 when the shaft 226 is in the extended position in comparison to when the shaft 226 is in the retracted position.

As illustrated in FIG. 2, the one or more electrical elements may include an electrosurgical electrode 228 (i.e., an electrode tip). In monopolar electrosurgery, the electrosurgical electrode 228 (active electrode) is placed at a desired surgical site and a return electrode (not shown) is placed somewhere else on the body of the patient. A practitioner can employ the shaft 226 to maneuver the electrosurgical electrode 228 around obstructions and/or through small spaces to reach a desired surgical site. The electrosurgical electrode 228 may be formed from a conductive material, such as stainless steel or other suitable metal. Although the electrosurgical electrode 228 may be configured as an electrode blade, the electrosurgical electrode 228 may be shaped alternatively as a needle, ball, etc., to suit a particular procedure. The one or more electrical elements may also include a light source 240 that delivers light via an optical structure 242 to make the surgical site more visible to the practitioner and/or imaging system. In some implementations, the light source 240 may include one or more light emitting diodes (LEDs) embedded in a printed circuit board (PCB).

The electrosurgical device 212 includes a power cord 222 that extends from the handle 224 to a proximal end 212a. The electrosurgical device 212 includes an electrical connector 221 disposed on the power cord 222 at the proximal end 212a. The electrical connector 221 can be inserted into, or otherwise coupled, to an electrosurgical generator (not shown) as described above. As illustrated, the electrical connector 221 may include one or more prongs that can be plugged into a connector of the electrosurgical generator.

The electrosurgical generator can generate an electrosurgical signal, such as a high-frequency alternating current, for the electrosurgical electrode 228. Additionally, the electrosurgical generator can provide electrical power to the light source 240. In general, the electrosurgical generator can provide any electrical signal (e.g., electrosurgical signal, electrical power, etc.) for the one or more electrical elements (e.g., electrosurgical electrode 228, light source 240, etc.).

The electrosurgical device 212 receives the electrosurgical signal and the electrical power via the electrical connector 221. The electrosurgical signal and the electrical power are then transmitted to the handle 224 through the power cord 222. The electrosurgical device 212 includes a first conducting element 236a that is integrated into the inner support structure 226c of the shaft 226 to deliver the electrosurgical signal from the handle 224 to the electrosurgical electrode 228. Additionally, electrosurgical device 212 includes a second conducting element 236b that is integrated into the inner support structure 226c of the shaft 226 to deliver the electrical power from the handle 224 to the light source 240.

The electrosurgical device 212 also includes one or more user input devices 230 (e.g., buttons, switches, etc.) disposed on the handle 224 to control the one or more electrical elements. In particular, the practitioner can employ the one or more user input devices 230 to allow the electrosurgical signal to be transmitted from electrosurgical generator to the electrosurgical electrode 228 or to allow the electrical power to be transmitted from the electrosurgical generator to the light source 240.

Upon transmission of the electrosurgical signal to the electrosurgical electrode 228, a current passes through the patient from the electrosurgical electrode 228 (active electrode) to the return electrode and generates heat at the surgical site according to monopolar electrosurgery: The electrosurgical generator can be controlled to determine the waveform of the electrosurgical signal. This waveform determines the corresponding tissue effects produced by the electrosurgical device 212. For monopolar electrosurgery, the first conducting element 236a may include a single conducting wire to deliver the electrosurgical signal to the electrosurgical electrode 228, while the second conducting element 236b may include two conducting wires to deliver the electrical power conventionally to the light source 240. Correspondingly, FIG. 2 illustrates three prongs for the electrical connector 221.

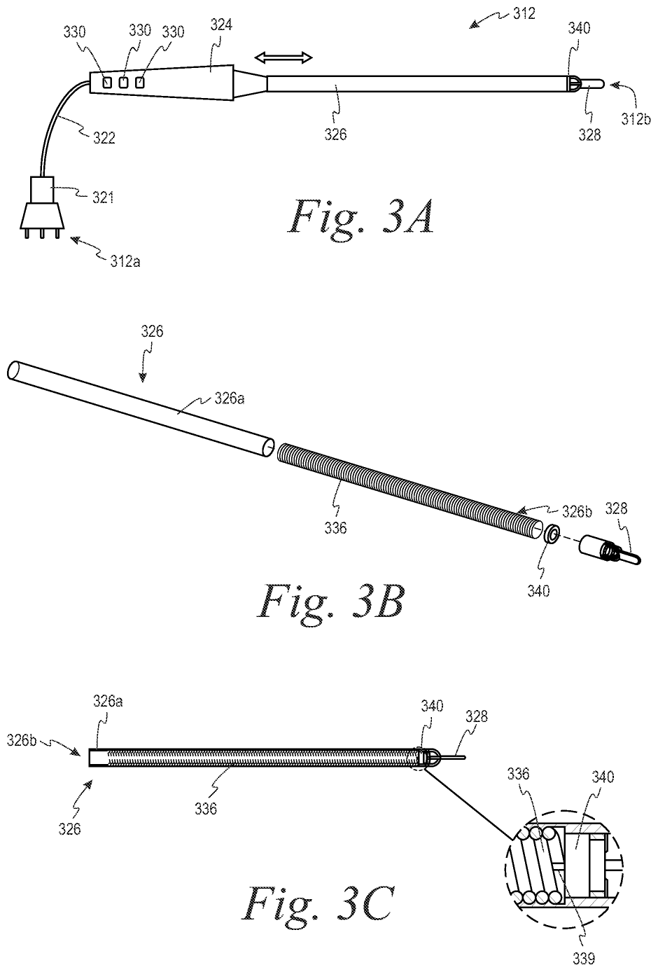

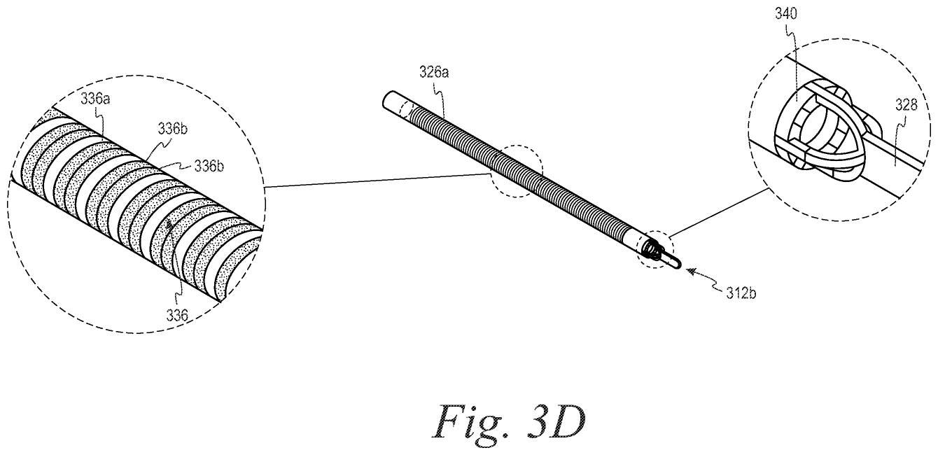

FIGS. 3A-D illustrate another electrosurgical device 312. The electrosurgical device 312 includes a handle 324 that allows a practitioner to manipulate the electrosurgical device 312. The electrosurgical device 312 includes a shaft 326 extending from the handle 324 to define a distal end 312b of the electrosurgical device 312. The electrosurgical device 312 includes one or more electrical elements coupled to the shaft 326 at the distal end 312b. The shaft 326 is configured to bend and position the one or more electrical elements relative to the handle 324.

As shown in FIGS. 3B-C, the shaft 326 includes an outer structure 326a defining a passageway 326b extending from the handle 324 to the one or more electrical elements. The shaft 326 also includes one or more conductors 336 extending through the passageway 326b to provide electrical signals from an electrosurgical generator (not shown) to the one or more electrical elements. The one or more conductors 336 combine to form a coil (a series of loops) that is configured to bend in response to a force that exceeds a threshold and to determine a bended shape for the shaft 326. Thus, in addition to transmitting electrical signals, the one or more conductors 336 also define an inner support structure for the shaft 326. As illustrated, the outer structure 326a may be formed from a heat shrink material (e.g., plastic) configured to shrink over the coil formed by the one or more conductors 336 (as a sheath). In this implementation, in comparison to the outer structure 326a, the one or more conductors 336 provide greater resistance to bending. Alternatively, the outer structure 326a may be a second coil defined by a second series of loops configured to allow bending of the shaft.

With the combination of the outer structure 326a and the one or more conductors 336, the shaft 326 is configured to reposition the one or more electrical elements relative to the handle 324. The shaft 326 is sufficiently flexible to take different shapes (e.g., a bended shape chosen by the practitioner), but is also sufficiently rigid to maintain a desired shaped during maneuvering and operation of the electrosurgical device 312.

In some implementations, the shaft 326 is further configured to move relative to the handle 324 from a retracted position to an extended position. As such, the shaft 326 extends the distal end 312b of the electrosurgical device 312 to a greater distance from the handle 324 when the shaft 326 is in the extended position in comparison to when the shaft 326 is in the retracted position.

As illustrated, the one or more electrical elements may include an electrosurgical electrode 328 (i.e., an electrode tip). In monopolar electrosurgery, the electrosurgical electrode 328 (active electrode) is placed at a desired surgical site and a return electrode (not shown) is placed somewhere else on the body of the patient. A practitioner can employ the shaft 326 to maneuver the electrosurgical electrode 328 around obstructions and/or through small spaces to reach the desired surgical site. The electrosurgical electrode 328 may be formed from a conductive material, such as stainless steel or other suitable metal. Although the electrosurgical electrode 328 may be configured as an electrode blade, the electrosurgical electrode 328 may be shaped alternatively as a needle, ball, etc., to suit a particular procedure. The one or more electrical elements may also include a light source 340 that delivers light to make the surgical site more visible to the practitioner and/or imaging system. As illustrated, the light source 340 may include a circular arrangement of LEDs (e.g., four LEDs) embedded in a PCB positioned proximally to the electrosurgical electrode 328.

The electrosurgical device 312 includes a power cord 322 that extends from the handle 324 to a proximal end 312a. The electrosurgical device 312 includes an electrical connector 321 disposed on the power cord 322 at the proximal end 312a. The electrical connector 321 can be inserted into, or otherwise coupled, to the electrosurgical generator. As illustrated, the electrical connector 321 may include one or more prongs that can be plugged into the electrosurgical generator.

The electrosurgical generator can generate an electrosurgical signal, such as a high-frequency alternating current, for the electrosurgical electrode 328. Additionally, the electrosurgical generator can provide electrical power to the light source 340. In general, the electrosurgical generator can provide any electrical signal (e.g., electrosurgical signal, electrical power, etc.) for the one or more electrical elements (e.g., electrosurgical electrode 328, light source 340, etc.).

The electrosurgical device 312 receives the electrosurgical signal and the electrical power via the electrical connector 321. The electrosurgical signal and the electrical power are then transmitted to the handle 324 through the power cord 322. The one or more conductors 336 include a first conducting element 336a that delivers the electrosurgical signal from the handle 324 to the electrosurgical electrode 328. Additionally, the one or more conductors 336 include a second conducting element 336b that delivers the electrical power from the handle 324 to the light source 340.

The electrosurgical device 312 also includes one or more user input devices 330 (e.g., buttons, switches, etc.) disposed on the handle 324 to control the one or more electrical elements. In particular, the practitioner can employ the one or more user input devices 330 to allow the electrosurgical signal to be transmitted from the electrosurgical generator to the electrosurgical electrode 328 or to allow the electrical power to be transmitted from the electrosurgical generator to the light source 340.

As shown generally in FIG. 3C, a conductor 336 can terminate at a connector 339 for an electrical element (e.g., the light source 340) at the distal end 312b. Accordingly, the connector 339 is in conductive communication with the electrical element and can transmit the appropriate electrical signals from the electrosurgical generator to the electrical element.

Upon transmission of the electrosurgical signal to the electrosurgical electrode 328, a current passes through the patient from the electrosurgical electrode 328 (active electrode) to the return electrode and generates heat at the surgical site according to monopolar electrosurgery: The electrosurgical generator can be controlled to determine the waveform of the electrosurgical signal. This waveform determines the corresponding tissue effects produced by the electrosurgical device 312. For monopolar electrosurgery; the first conducting element 336a may include a single conducting wire to deliver the electrosurgical signal to the electrosurgical electrode 328, while the second conducting element 336b may include two conducting wires to deliver the electrical power conventionally to the light source 340. Correspondingly; FIG. 3A illustrates three prongs for the electrical connector 321.

As shown in FIG. 3D, the conducting wire (insulated) for the first conducting element 336a and the two conducting wires (insulated) for the second conducting element 336b spiral in combination to form the coil which provides the inner support structure for the shaft 326. In some implementations, different materials may be employed for the three wires to produce a blend of different desired material properties. As such, the shaft 326 can be configured to have properties that suit a particular use of the electrosurgical device 312. For instance, one of the wires can be more ductile to tolerate a larger plastic deformation, while the other two wires can be more rigid to help the shaft 326 maintain a desired shape. Furthermore, one or more strands of additional material, such as plastics, may be combined with the conductors 336 to further determine the properties of the shaft 326.

Although the one or more conductors 336 described above may form a coil defined by a series of loops, other implementations may employ one or more conductors that form different supporting shapes. The one or more conductors may include one or more wires that provide transverse support for the outer structure 326a without forming a coil, e.g., by forming a series of truss-like supports within the passageway 326b. Furthermore, the one or more conductors may provide support along the entire length of the shaft 326 or at specific segments of the shaft 326.

As described above, the one or more conductors 336 may include the first conductor and the second conductor, where the one or more conductors 336 include a first coil that extends into conductive communication with the electrosurgical electrode 328 at the distal end 312b and a second coil that extends into conductive communication with the light source 340.

Although the features above may be described in relation to electrosurgery; it is understood that such features can be more generally implemented in any medical instrument for any medical procedure. The shaft of any medical instrument may include an outer structure and a supporting inner structure, where the inner structure may be a coil formed from one or more conductors that transmit electrical signals to one or more electrical elements disposed on an end of the shaft.

Furthermore, although the inner structures above are formed from conductors, it is understood that inner structures may be formed from other elements. For instance, inner structures may be alternatively or additionally formed from optical fibers that transmit light or optical signals to or from an end of the shaft.

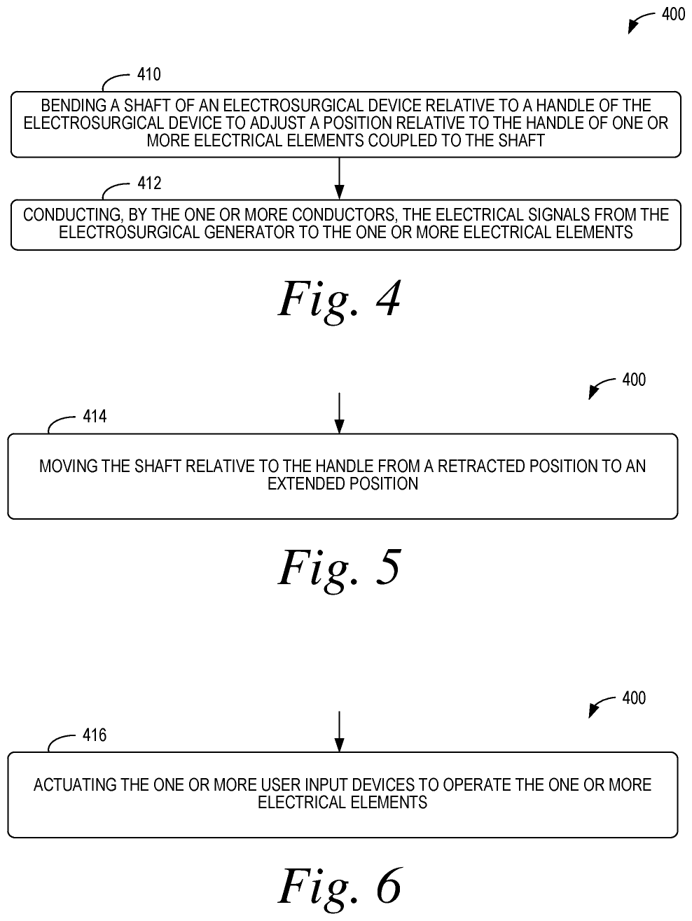

Referring now to FIG. 4, a flowchart of a method 400 of operating an electrosurgical device is shown according to an example. At block 410, the method 400 includes bending a shaft of an electrosurgical device relative to a handle of the electrosurgical device to adjust a position relative to the handle of one or more electrical elements coupled to the shaft. The shaft extends from the handle to define a distal end of the electrosurgical device. The one or more electrical elements are coupled to the shaft at the distal end of the electrosurgical device. The shaft includes: (i) an outer structure defining a passageway extending from the handle to the one or more electrical elements, and (ii) one or more conductors extending through the passageway to transmit electrical signals from an electrosurgical generator to the one or more electrical elements. The one or more conductors form a coil configured to bend in response to greater than a threshold amount of force and to determine a bended shape for the shaft. In an example, bending the shaft can include applying a force greater than the threshold amount of force. At block 412, the method 400 can also include conducting, by the one or more conductors, the electrical signals from the electrosurgical generator to the one or more electrical elements.

FIGS. 5-6 depict additional aspects of the method 400 according to further examples. As shown in FIG. 5, the method 400 can also include moving the shaft relative to the handle from a retracted position to an extended position at block 414. The shaft extends the distal end of the electrosurgical device to a greater distance from the handle when the shaft is in the extended position in comparison to when the shaft is in the retracted position.

In the example shown in FIG. 6, the handle includes one or more user input devices configured to operate the one or more electrical elements. Additionally, in FIG. 5, the method further includes actuating the one or more user input devices to operate the one or more electrical elements at block 416.

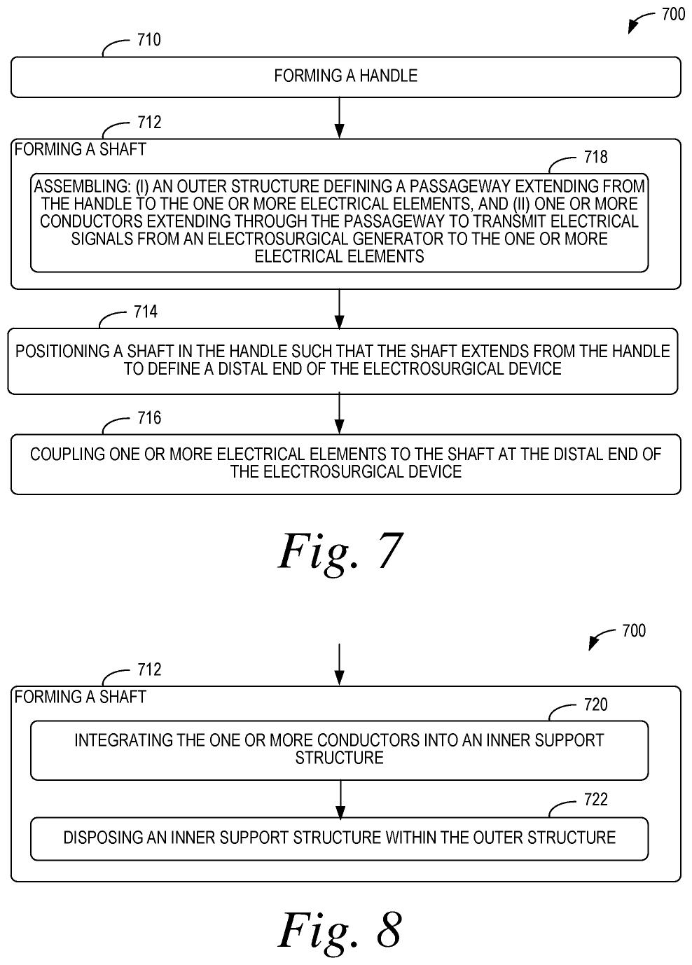

FIG. 7 depicts a flowchart for a method 700 of forming an electrosurgical device, according to an example. The method 700 includes forming a handle at block 710, forming a shaft at block 712, positioning a shaft in the handle such that the shaft extends from the handle to define a distal end of the electrosurgical device at block 714, and coupling one or more electrical elements to the shaft at the distal end of the electrosurgical device at block 716. The shaft is configured to bend and position the one or more electrical elements relative to the handle. Forming the shaft at block 712 can include assembling: (i) an outer structure defining a passageway extending from the handle to the one or more electrical elements, and (ii) one or more conductors extending through the passageway to transmit electrical signals from an electrosurgical generator to the one or more electrical elements at block 718. The one or more conductors form a coil configured to bend in response to a force that exceeds a threshold and to determine a bended shape for the shaft.

FIG. 8 depicts additional aspects of the method 500 according to a further example. In FIG. 8, forming the shaft at block 712 can include: integrating the one or more conductors into an inner support structure at block 720, and disposing an inner support structure within the outer structure at block 722. The inner support structure is configured to bend with the outer structure and transversely support the outer structure from within and along the passageway.

The description of the different advantageous arrangements has been presented for purposes of illustration and description, and is not intended to be exhaustive or limited to the embodiments in the form disclosed. Many modifications and variations will be apparent to those of ordinary skill in the art. Further, different advantageous embodiments may provide different advantages as compared to other advantageous embodiments. The implementation or implementations selected are chosen and described in order to best explain the principles of the embodiments, the practical application, and to enable others of ordinary skill in the art to understand the disclosure for various embodiments with various modifications as are suited to the particular use contemplated.

What is claimed is:

1. An electrosurgical device, comprising:
a handle;
a shaft extending from the handle to define a distal end of the electrosurgical device; and
one or more electrical elements coupled to the shaft at the distal end of the electrosurgical device,
wherein the shaft is configured to bend and position the one or more electrical elements relative to the handle, the shaft including:
an outer structure defining a passageway extending from the handle to the one or more electrical elements; and
one or more conductors extending through the passageway to transmit electrical signals from an electrosurgical generator to the one or more electrical elements, wherein the one or more conductors form a coil configured to bend in response to a force that exceeds a threshold and to determine a bended shape for the shaft.

2. The electrosurgical device of claim 1, wherein the shaft is further configured to move relative to the handle from a retracted position to an extended position, the shaft extending the distal end of the electrosurgical device to a greater distance from the handle when the shaft is in the extended position in comparison to when the shaft is in the retracted position.

3. The electrosurgical device of claim 1, wherein the one or more electrical elements include at least one of an electrosurgical electrode or a light source.

4. The electrosurgical device of claim 1, wherein the outer structure is formed from a heat shrink material configured to shrink over the coil formed by the one or more conductors.

5. The electrosurgical device of claim 1, wherein the outer structure is a second coil defined by a second series of loops configured to allow bending of the shaft.

6. The electrosurgical device of claim 1, wherein the one or more conductors form the coil with one or more strands of additional material to determine the threshold for bending the coil.

7. The electrosurgical device of claim 1, wherein the handle includes one or more user input devices configured to operate the one or more electrical elements.

8. The electrosurgical device of claim 1, further comprising an electrical connector extending proximally from the handle and configured to receive the electrical signals from the electrosurgical generator and for transmission to the one or more conductors.

9. The electrosurgical device of claim 1, wherein the one or more conductors includes conducting wire.

10. The electrosurgical device of claim 1, wherein, in comparison to the outer structure, the coil formed by the one or more conductors provides greater resistance to bending.

11. An electrosurgical device, comprising:
a handle;
a shaft extending from the handle to define a distal end of the electrosurgical device; and
one or more electrical elements coupled to the shaft at the distal end of the electrosurgical device,
wherein the shaft is configured to bend and position the one or more electrical elements relative to the handle, the shaft including:
an outer structure defining a passageway extending from the handle to the one or more electrical elements; and
one or more conducting wires extending through the passageway to provide electrical signals from an electrosurgical generator to the one or more electrical elements, wherein the one or more conducting wires form a shape configured to bend in response to a force that exceeds a threshold and to determine a bended shape for the shaft.

12. The electrosurgical device of claim 11, wherein the shaft is further configured to move relative to the handle from a retracted position to an extended position, the shaft extending the distal end of the electrosurgical device to a greater distance from the handle when the shaft is in the extended position in comparison to when the shaft is in the retracted position.

13. The electrosurgical device of claim 11, wherein the one or more electrical elements include at least one of an electrosurgical electrode or a light source.

14. The electrosurgical device of claim 11, wherein the outer structure is formed from a heat shrink material configured to shrink over the shape formed by the one or more conducting wires.

15. The electrosurgical device of claim 11, wherein the outer structure is a coil defined by a series of loops configured to allow bending of the shaft.

16. The electrosurgical device of claim 11, wherein the one or more conducting wires form the shape with one or more strands of additional material to determine the threshold for bending the shape.

17. The electrosurgical device of claim 11, further comprising an electrical connector extending proximally from the handle and configured to receive the electrical signals from the electrosurgical generator and for transmission to the one or more conducting wires.

18. The electrosurgical device of claim 11, wherein the handle includes one or more user input devices configured to operate the one or more electrical elements.

19. The electrosurgical device of claim 11, wherein, in comparison to the outer structure, the shape formed by the one or more conducting wires provides greater resistance to bending.

20. An electrosurgical device, comprising:
a handle;
a shaft extending from the handle to define a distal end of the electrosurgical device; and
one or more electrical elements coupled to the shaft at the distal end of the electrosurgical device,
wherein the shaft is configured to bend in response to a force that exceeds a threshold and thereby reposition the one or more electrical elements relative to the handle, the shaft including:
an outer structure defining a passageway extending from the handle to the one or more electrical elements; and
an inner support structure disposed within the outer structure and configured to bend with the outer structure and transversely support the outer structure from within and along the passageway, wherein one or more conductors are integrated into the inner support structure, the one or more conductors configured to provide electrical signals from an electrosurgical generator to the one or more electrical elements.

21. The electrosurgical device of claim 20, wherein the shaft is further configured to move relative to the handle from a retracted position to an extended position, the shaft extending the distal end of the electrosurgical device to a greater distance from the handle when the shaft is in the extended position in comparison to when the shaft is in the retracted position.

22. The electrosurgical device of claim 20, wherein the one or more electrical elements include at least one of an electrosurgical electrode or a light source.

23. The electrosurgical device of claim 20, wherein the outer structure is formed from a heat shrink material configured to shrink over the inner support structure.

24. The electrosurgical device of claim 20, wherein the inner support structure is a coil defined by a series of loops configured to bend in response to the force that exceeds the threshold and determine a bended shape for the shaft.

25. The electrosurgical device of claim 20, wherein the outer structure is a coil defined by a series of loops configured to allow bending of the shaft.

26. The electrosurgical device of claim 20, further comprising an electrical connector extending proximally from the handle and configured to receive the electrical signals from the electrosurgical generator and for transmission to the one or more conductors.

27. The electrosurgical device of claim 20, wherein the handle includes one or more user input devices configured to operate the one or more electrical elements.

28. The electrosurgical device of claim 20, wherein the one or more conductors include conducting wire.

29. The electrosurgical device of claim 20, wherein, in comparison to the outer structure, the inner support structure provides greater resistance to bending.

30. A method of operating an electrosurgical device, comprising:
bending a shaft of an electrosurgical device relative to a handle of the electrosurgical device to adjust a position, relative to the handle, of one or more electrical elements coupled to the shaft, wherein the shaft extends from the handle to define a distal end of the electrosurgical device, wherein the one or more electrical elements are coupled to the shaft at the distal end of the electrosurgical device,
wherein the shaft comprises: (i) an outer structure defining a passageway extending from the handle to the one or more electrical elements, and (ii) one or more conductors extending through the passageway to transmit electrical signals from an electrosurgical generator to the one or more electrical elements,
wherein the one or more conductors form a coil configured to bend in response to greater than a threshold amount of force and to determine a bended shape for the shaft, wherein bending the shaft comprises applying a force greater than the threshold amount of force; and
conducting, by the one or more conductors, the electrical signals from the electrosurgical generator to the one or more electrical elements.

31. The method of claim 30, wherein the shaft further comprises an inner support structure disposed within the outer structure and configured to bend with the outer structure and transversely support the outer structure from within and along the passageway, wherein the one or more conductors are integrated into the inner support structure.

32. The method of claim 30, further comprising moving the shaft relative to the handle from a retracted position to an extended position, the shaft extending the distal end of the electrosurgical device to a greater distance from the handle when the shaft is in the extended position in comparison to when the shaft is in the retracted position.

33. The method of claim 30, wherein the one or more electrical elements include at least one of an electrosurgical electrode or a light source.

34. The method of claim 30, wherein the outer structure is formed from a heat shrink material configured to shrink over the coil formed by the one or more conductors.

35. The method of claim 30, wherein the outer structure is a second coil defined by a second series of loops configured to allow bending of the shaft.

36. The method of claim 30, wherein the one or more conductors form the coil with one or more strands of additional material to determine the threshold amount of force for bending the coil.

37. The method of claim 30, wherein the handle includes one or more user input devices configured to operate the one or more electrical elements, and wherein the method further comprises actuating the one or more user input devices to operate the one or more electrical elements.

38. The method of claim 30, further comprising receiving, by an electrical connector extending proximally from the handle, the electrical signals from the electrosurgical generator.

39. A method of forming an electrosurgical device, comprising:
forming a handle;
forming a shaft;
positioning a shaft in the handle such that the shaft extends from the handle to define a distal end of the electrosurgical device; and
coupling one or more electrical elements to the shaft at the distal end of the electrosurgical device,
wherein the shaft is configured to bend and position the one or more electrical elements relative to the handle,
wherein forming the shaft comprises assembling: (i) an outer structure defining a passageway extending from the handle to the one or more electrical elements, and (ii) one or more conductors extending through the passageway to transmit electrical signals from an electrosurgical generator to the one or more electrical elements,
wherein the one or more conductors form a coil configured to bend in response to a force that exceeds a threshold and to determine a bended shape for the shaft.

40. The method of claim 39, wherein forming the shaft further comprises:
integrating the one or more conductors into an inner support structure; and
disposing an inner support structure within the outer structure, wherein the inner support structure is configured to bend with the outer structure and transversely support the outer structure from within and along the passageway.
